(19) United States
(12) Patent Application Publication
(10) Pub. No.: US 2006/0287627 A1
(43) Pub. Date: Dec. 21, 2006

(54) System and method for patient specific spinal therapy

(75) Inventor: Scot Johnson, Lutz, FL (US)

Correspondence Address: MCANDREWS HELD & MALLOY, LTD, 500 WEST MADISON STREET, SUITE 3400, CHICAGO, IL, 60661, US

(73) Assignee: Axiom Worldwide, Inc., Tampa, FL (US)

(21) Appl. No.: 11/452,008

(22) Filed: Jun. 13, 2006

Related U.S. Application Data
(60) Provisional application No. 60/690,962, filed on Jun. 16, 2005.
(60) Provisional application No. 60/723,200, filed on Oct. 3, 2005.

(51) Int. Cl. A61F 5/00

(52) U.S. Cl. 602/33

(57) ABSTRACT

A system for applying a patient-specific treatment profile during spinal therapy. The system includes a control system for implementing a spinal treatment profile, an actuator for producing a tensile force as defined in the treatment profile based on commands from the control system, a patient interface device configured to apply the tensile force from the actuator to the spine of a patient, and a feedback system configured to provide a metric resulting from the application of the tensile force to the spine to the control system, wherein the treatment profile includes a limit based on the metric related to the application of the tensile force to the spine such that, when the application of the tensile force to the spine results in a metric that exceeds the limit, the control system reduces the tensile force applied by the actuator to the spine.

### RELATED APPLICATIONS

This application is related to, and claims priority from, Provisional Application No. 60/690,962, filed Jun. 16, 2005, titled “System And Method For Patient Specific Spinal Decompression Therapy,” the complete subject matter of which is incorporated herein by reference in its entirety. This application is related to, and claims priority from, Provisional Application No. 60/723,200, filed Oct. 3, 2005, titled “System And Method For Patient Specific Spinal Therapy,” the complete subject matter of which is incorporated herein by reference in its entirety.

### BACKGROUND OF THE INVENTION

The present invention relates to a spinal therapy system. More specifically, the present invention relates to a system that uses tension profiles for applying tension to a patient's spine to treat the spine and that monitors and adjusts the treatment based on metrics specific to the patient's response to the treatment.

Therapists utilize spinal decompression therapy to treat various spinal ailments including herniated discs, degenerative disc disease, sciatica, posterior facet syndrome, and post surgical pain. Decompression therapy is a derivative of traction-based therapy, whereby the spine is placed into a state of tension by an outside force (such as by a therapist manually or by an automated process). The spine is typically held in a continuous state of tension during traction-based therapy. Decompression therapy differs from traction therapy in that the traction is applied to the spine at a specific angle. Also, during decompression therapy, various tensile forces are applied or cycled throughout the treatment period according to non-linear, curvilinear, sinusoidal, exponential, or logarithmic mathematical functions such that para-spinal muscles are relaxed and fatigued, allowing for inter-discal separation. These functions provide for a smooth transition between different levels of tension. In either traditional traction or decompression therapy, spinal tension is typically maintained for periods of 30 minutes or longer.

As the spine is placed into a state of tension, the spinal vertebrae are separated in order to allow the intervertebral discs to realign into their proper positions. This action allows herniated discs time to heal in a non-loaded state. Additionally, nutrient-rich spinal fluid (nucleus pulposa) is drawn to the sites of tension via the pressure drop created by the separation of the vertebrae. However, para-spinal muscles may react involuntarily to the “stretching” of the spine by tensing in opposition to the force. Also, the conscious human patient may voluntarily and/or subconsciously flex the spinal muscles in reaction to tensile forces during the traction. Either or both patient reactions degrade the effectiveness of spinal therapy.

One common spinal decompression therapy system utilizes a non-feedback-providing electromechanical actuator (or any type of pneumatic, magnetic, hydraulic, or chemical actuator) connected to a patient via a patient interface device, such as a tension strap. The speed or rate of operation of the actuator is increased or decreased to produce resultant tension changes at the point where the strap is attached to the patient. The system also includes a tension measuring device (e.g., a loadcell) that is connected inline with the tension-producing actuator and patient to communicate tension metrics to a tension-producing actuator controlling device (e.g. a computer). Thus, the system operates as a controlled-feedback loop whereby a planned tension profile can be applied to the patient and the actual applied forces can be verified by the computer.

All traction and decompression therapy systems utilize tension-producing actuator speeds or rates to increase or decrease the tension applied to the patient and measure the tension via a tension measurement device. In other words, currently available traction and decompression systems utilize a speed or rate based algorithm to create spinal decompression. While this methodology can elicit successful treatment of some spinal ailments, it has disadvantages. For example, tension-producing actuator speeds or rates are measured by a tension measurement device to provide feedback that can be measured and recorded. These recordings can be recalled during future therapy sessions but can only show whether or not the patient completed an intended tension profile, i.e., show the tension treatment the patient received during the session. These recordings from a tension profile do not provide any information on the state of the patient's spine during treatment. Spinal elongation is a more appropriate metric for measuring the success of decompression therapy than tension-providing actuator speed or rate. Intervertebral discs are allowed to heal when intervertebral disc space is increased. As decompression therapy is a localized effect, there is a select region of the spine that can be elongated. Thus, if the tension-producing actuator provides its controller with feedback related to spinal column elongation, then the spinal elongation metric can be used to track spinal column relaxation and accommodate the therapy to the patient's reactions to elongation.

Furthermore, the typical spinal decompression system does not allow for measuring resistance of the spine to tension or the reaction of the para-spinal muscles to tension. In other words, the typical decompression system does not allow therapists to account for how much resistance to the tension treatment is due to spinal resistance and how much is to para-spinal muscle resistance. Thus, the therapist cannot adjust the tension during treatment to accommodate resistance from either the spine or the para-spinal muscles. Also, the typical spinal decompression system does not allow the therapist to monitor psychological para-spinal muscle constriction due to the tension and alter the tension accordingly.

Therefore, a need exists for a spinal decompression system that overcomes the deficiencies of conventional systems.

### BRIEF SUMMARY OF THE INVENTION

Certain embodiments of the present invention include a system for applying a treatment profile during spinal therapy. The system includes a control system for implementing a spinal treatment profile, an actuator for producing a tensile force based on the implementation of the spinal treatment profile by the control system, a patient interface device configured to apply the tensile force from the actuator to the spine of a patient, and a feedback system configured to provide metrics resulting from the application of the tensile force to the spine to the control system such that the control system calculates elongation of the spine of the patient as a result of the tensile force applied to the spine and monitors the elongation relative to an intended elongation program of the treatment profile.

Certain embodiments of the present invention include a system for applying a treatment profile during spinal therapy. The system includes a control system for implementing a spinal treatment profile, an actuator for producing a tensile force based on the implementation of the spinal treatment profile by the control system, a patient interface device configured to apply the tensile force from the actuator to the spine of a patient, and a feedback system configured to provide metrics resulting from the application of the tensile force to the spine to the control system such that the control system calculates work performed by the actuator during application of the treatment profile and monitors the performed work relative to predetermined work limit of the treatment profile.

Certain embodiments of the present invention include a system for applying a treatment profile during spinal therapy. The system includes a control system for implementing a spinal treatment profile, an actuator for producing a tensile force as defined in the treatment profile based on commands from the control system, a patient interface device configured to apply the tensile force from the actuator to the spine of a patient, and a feedback system configured to provide a metric resulting from the application of the tensile force to the spine to the control system, wherein the treatment profile includes a limit based on the metric related to the application of the tensile force to the spine such that, when the application of the tensile force to the spine results in a metric that exceeds the limit, the control system reduces the tensile force applied by the actuator to the spine.

Certain embodiments of the present invention include a method of applying a treatment profile for spinal therapy. The method includes providing a tension-producing actuator, providing a control system that implements the treatment profile to control the actuator, applying a tensile force to the spine of a patient in accordance with the treatment profile, calculating the elongation of the spine of the patient resulting from the application of the tensile force, and monitoring and adjusting the application of the tensile force applied to the spine of the patient with the control system based on the calculations of spinal elongation during therapy.

Certain embodiments of the present invention include a method of applying a treatment profile for spinal therapy. The method includes providing a tension-producing actuator, providing a control system that implements the treatment profile to control the actuator, applying a tensile force to the spine of a patient with the actuator in accordance with the treatment profile, calculating the work performed by the actuator in applying the tensile force to the spine of the patient, and monitoring and adjusting the application of the tensile force applied to the spine of the patient with the control system based on the calculations of work performed by the actuator during the spinal therapy.

### BRIEF DESCRIPTION OF THE DRAWINGS

FIG. 1 illustrates a side view of a spinal therapy system formed according to an embodiment of the present invention.

FIG. 2 illustrates an isometric view of a tension-producing actuator formed according to an embodiment of the present invention.

FIG. 3 illustrates an isometric view of a tension-producing actuator formed according to an embodiment of the present invention.

FIG. 4 illustrates an isometric view of a tension measurement apparatus of FIG. 1 formed according to an embodiment of the present invention.

FIG. 5 illustrates an isometric view of a tension-producing actuator controller of FIG. 1 formed according to an embodiment of the present invention.

FIG. 6 illustrates an isometric view of a bed formed according to an embodiment of the present invention.

FIG. 7 illustrates an isometric view of the lower portion of the bed of FIG. 6 extended on a shaft.

FIG. 8 illustrates a tension profile for spinal decompression.

FIG. 9 illustrates a spinal elongation profile for spinal therapy formed according to an embodiment of the present invention.

FIG. 10 illustrates the spinal elongation profile of FIG. 9 showing where the intended elongation was adjusted because the monitored tensile forces exceeded the preset maximum limits set for that portion of the spinal elongation profile.

FIG. 11 illustrates a flowchart demonstrating an algorithm for implementing patient specific spinal therapy using a distance-based spinal elongation methodology formed according to an embodiment of the present invention.

FIG. 12 illustrates a tension profile for spinal therapy formed according to an embodiment of the present invention.

FIG. 13 illustrates a tension profile for spinal therapy formed according to an embodiment of the present invention.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentalities shown in the attached drawings.

### DETAILED DESCRIPTION OF THE INVENTION

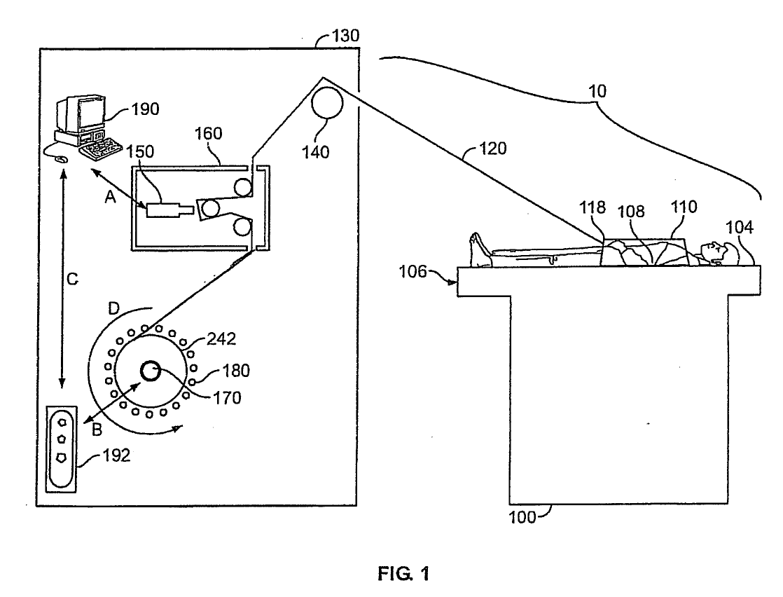

FIG. 1 illustrates a spinal therapy system 10 used to treat a patient 110 formed according to an embodiment of the present invention. The system 10 includes a microprocessor, control system, or computing device 190 having firmware and/or software that operates to utilize and control an actuator 170. The computing device 190 is configured to interface with a user, such as by use of a monitor and keyboard setup. By way of example only, the actuator 170 may be electronically, hydraulically, pneumatically, or mechanically operated. The actuator 170 is connected to a patient 110 via a patient interface device 120. By way of example, the actuator 170 may be operated through a system of gears or pulleys such that the tensile forces applied to the patient 110 by the patient interface device 120 are carefully controlled. This system 10 is used to perform decompression therapy on the patient 110 by applying cycles of tensile forces from the actuator 170 on the spine 108 of the patient 110 through the interface device 120. Alternatively, the system 10 may be used to perform traction therapy without use of cycles of tensile forces.

The patient 110 is positioned on a mechanical apparatus 100 that may be a flat surface such as a bed or table. The bed 100 includes a head end 104 where the patient 110 lies his or her head and a base end 106 where the patient 110 lies his or her legs and feet. The bed 100 is positioned such that the patient 110 may be easily placed into alignment for treatment with the system 10. Additionally, the bed 100 may employ arm supports or rails to position the patient 110. The patient 110 wears a harness 118 that is connectable to the patient interface device 120. Alternatively, the patient may wear any other appropriate device that is configured to connect the patient 110 to the interface device 120. The interface device 120 may be a strap, belt, or cable that is positioned relative to the patient 110 via a patient interface positioning device 140. The patient interface positioning device 140 may itself be moved to preferred positions by additional actuators. The harness 118 is connected to the actuator 170 by the patient interface device 120. The harness 118 may be connected to the patient interface device 120 through a clip or buckle that may alternately be secured and removed. The interface device 120 is configured to deliver and align tensile forces generated by the actuator 170 through the harness 118 along the spine 108 of the patient 110.

The system 10 further includes a tensile force feedback system 160 which engages the interface device 120 between the actuator 170 and the harness 118. The feedback system 160 may include a loadcell or dynamometer 150 that is positioned inline with the actuator 170 and is configured for electronically providing feedback to the computing device 190 as indicated by arrow A. The actuator 170 electronically communicates with, and is controlled directly by, an actuator controller 192 as shown by arrow B. By way of example only the actuator controller 192 is a servo-amplifier 192. The actuator 170 may also be attached to, or connected inline with, an encoder 180 that is capable of communicating motor shaft position and other motor metrics with the servo-amplifier 192. The servo-amplifier 192 may be capable of calculating any number of motor metrics, including work, position, distance, and rate and electronically communicating those metrics to, and receiving them from, the computing device 190 as indicated by arrow C to the computing device 190.

The computing device 190 may be configured to communicate with the servo-amplifier 192, and the actuator 170, to monitor and to correct as needed the resultant tensile force and motor metrics applied by the actuator 170 from the servo-amplifier 192. The computing device 190 may also be configured for use with a user interface system (e.g., keyboard and monitor) which communicates and deciphers the user's commands to the computer 190. This interface allows the user to structure treatment parameters. By way of example, all tension-producing and delivery apparati are contained within a tower 130 located in a position relative to the patient 110.

In operation, spinal treatment begins by positioning the patient 110 correctly onto the bed 100. The patient's head is positioned at the head end 104 of the bed 100, and the patient's feet are positioned at the base end 106 of the bed 100. The patient 110 is outfitted with the harness 118 such that the patient 110 is connected to the patient interface device 120, and the harness 118 is configured to apply tensile forces to the spine 108 of the patient 110. The operator of the decompression system 10 may use the patient interface system of the computer 190 to select the proper treatment parameters for the therapy.

The operator may then select a tension treatment program for the patient 110 and instruct the computing device 190 to execute the selected treatment profile. The computing device 190 activates the servo-amplifier 192 and/or actuator 170 such that the actuator 170 rotates, for example in the direction of arrow D, to tighten the patient interface device 120 and thus apply tension to the patient's spine 108 through the harness 118. The computing device 190 adjusts the tensile output to follow the cycles of tensile forces defined in the treatment program entered by the user. The program may include low and high tension plateaus above, by way of example only, 125 pounds, and may also include any number of decompression therapy variations cyclically applying tension to the patient's spine 108.

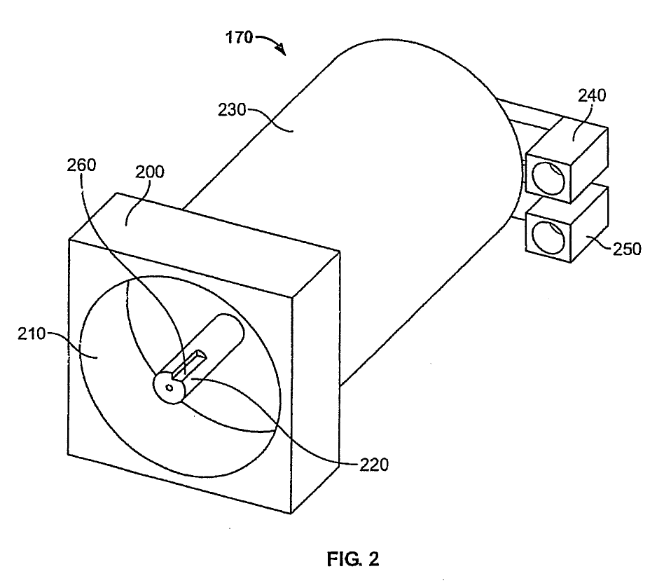

FIG. 2 illustrates an isometric view of a tension-producing actuator 170 for use with the system 10 of FIG. 1. The actuator 170 includes a servo-motor 230 with an accompanying motor encoder device 200. The motor encoder device 200 includes sensory devices 210 that monitor the movement of a motor shaft 220. The servo-motor 230 and encoder 200 communicate motor shaft 220 movement via a port 250 that is connected to the actuator controller 192 and/or the computing device 190 (FIG. 1). Power for the servo-motor 230 may be transferred via a port 240 connected to the servo-amplifier 192 and/or the computing device 190.

A spool 242 (FIG. 1) is mounted to the motor shaft 220 and locked in position on the shaft 220 via a locking notch 260. The patient interface device 120 (FIG. 1) is wound about the spool 242 and thus the shaft 220. When the servo motor 230 is activated, the shaft 220 of the servo-motor 230 rotates and tension is increased or decreased in the strap 120, depending on the direction the spool 242 rotates, and thus at the point where the patient interface device 120 is connected to the patient 110 (FIG. 1). The distance the motor 230 rotates is controlled by the encoder 200, which communicates the rotational distance the shaft 220 rotates to the actuator controller 192 (FIG. 1) and/or the computing device 190 (FIG. 1). Also, the work performed by the servo motor 230 may be communicated to the encoder 200 and to the actuator controller 192 and/or the computing device 190. These metrics may be used to employ the distance-based spinal decompression methodology of the present invention. Alternatively, the distance the motor 230 rotates may be determined by other devices besides the encoder such as by optical measuring devices.

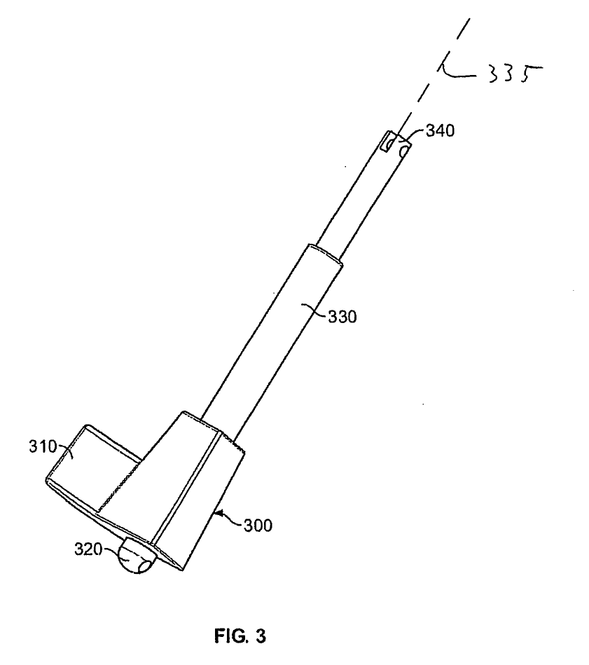

FIG. 3 illustrates an isometric view of an alternative embodiment of the tension-producing actuator 170 of FIG. 1 that is a linear actuator 300 with an integrated motor encoder device 310. The linear actuator 300 retracts or extends a motor shaft 330 linearly, typically along a single axis 335. The linear actuator 300 includes a motor clevis 320 that allows the actuator 300 to rotate about a connection to the system 10.

Like the actuator 170 of FIG. 2, the actuator 300 of FIG. 3 may be used in conjunction with the patient interface device 120 (FIG. 1) to deliver tensile forces to the patient 110 (FIG. 1). The strap 120 may be connected to the end of the motor shaft 330 at a motor shaft clevis 340. As the linear actuator 300 retracts and extends the motor shaft 330, tensile forces are communicated to the patient 110 along the harness 118 (FIG. 1). The motor encoder device 310 deciphers and communicates linear actuator metrics, which may include the travel distance of the motor shaft 330 and work, to the actuator controller 192 and/or the computing device 190 (FIG. 1). These metrics can be used to employ the distance-based spinal decompression methodology of the present invention.

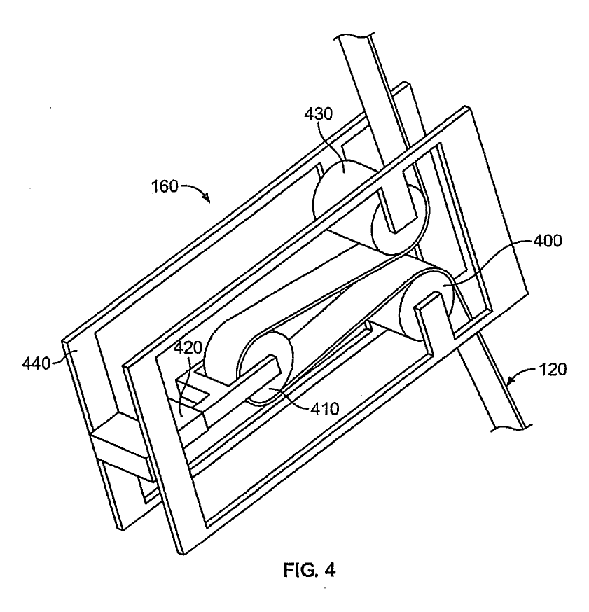

FIG. 4 illustrates an isometric view of a tension measurement apparatus 160 for use with the system 10 of FIG. 1. The tension measurement apparatus 160 is configured for use with the patient interface device 120. The tension measurement apparatus 160 includes a housing 440 that contains a specific configuration of devices which gather tensile force data between the tension-producing actuator 170 (FIG. 1) and the patient 110 (FIG. 1).

The strap 120 is connected to the tension-producing actuator 170 as shown in FIG. 1. By way of example, at a point between where the strap 120 is connected to the actuator 170 and the patient 110, the strap 120 enters the tension measurement apparatus 160 by extending over an entry roller 400 through a tension measurement device pulley 410. The strap 120 extends back and under an exit roller 430 and out of the tension measurement apparatus 160.

The tension measurement device pulley 410 is connected to a loadcell 420, or any number of other devices that measure force. The entry roller 400 and exit roller 430 position the strap 120 in-line with the tension measurement device pulley 410. As the tension-producing actuator 170 (FIG. 1) retracts the strap 120 and the patient's body 110 opposes the strap retraction, the strap 120 pulls against the tension measurement device pulley 410. The loadcell 420 is connected to the tension measurement device pulley 410 such that the loadcell 420 takes an accurate measurement of the tensile forces that the strap 120 undertakes.

The loadcell 420 may communicate tensile force measurement to the tension-producing actuator controller 192 (FIG. 1) and/or to the computing device 190 (FIG. 1). This feedback from the loadcell 420 can be used to monitor and calculate treatment settings.

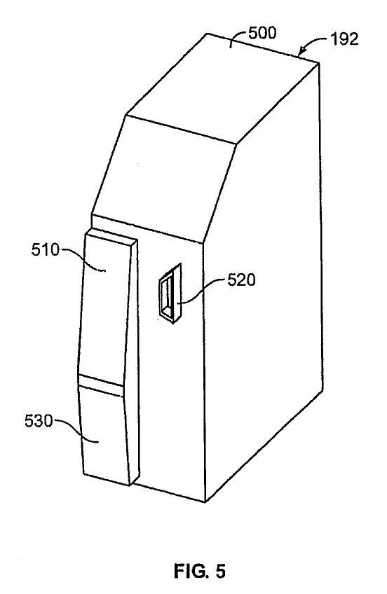

FIG. 5 illustrates an isometric view of a tension-producing actuator controller 192 for use with the system of FIG. 1. The tension-producing actuator controller 192 may be a servo controller 500. The servo controller 500 communicates power to the actuator 170 (FIG. 1) via power outlets 510 that are connected to the actuator 170. The servo controller 500 receives tension-producing actuator metrics (such as position, power consumption, direction, etc.) that are generated by either the actuator 170 (FIG. 1) itself or by an integrated or separate encoder device via communication connections 530. The servo controller 500 communicates actuator information and actuator profile information with the computing device 190 (FIG. 1) via a communications port 520.

The servo controller 500 may communicate actuator metrics with the computer 190 (FIG. 1) and actuator profile data and power with the actuator 170 (FIG. 1). Precise adjustment of the actuator 170 produces precise changes in the extension or retraction of the patient interface device 120 (FIG. 1). This kind of control loop makes possible a decompression system where a profile based on metrics such as spinal elongation or motor work may be implemented.

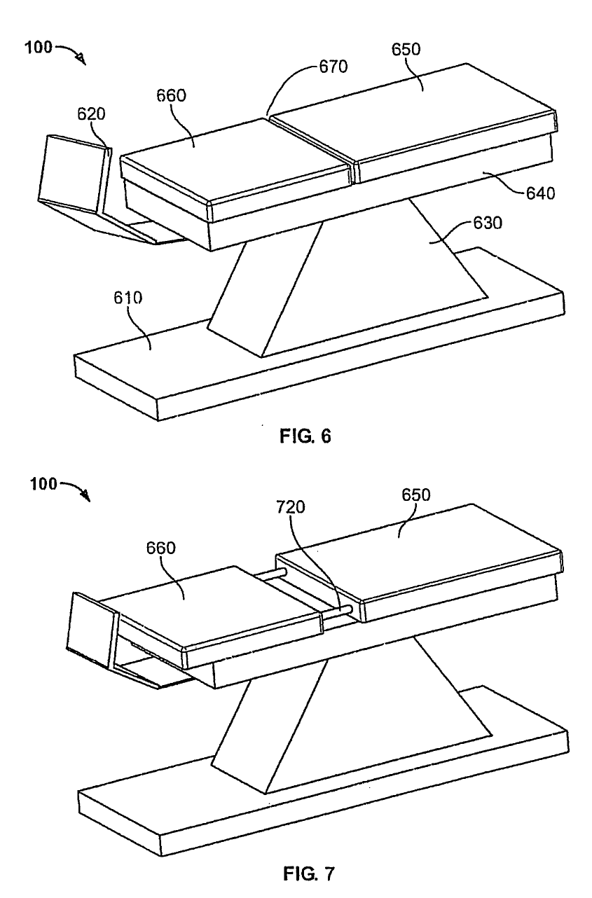

FIG. 6 illustrates an isometric view of a bed 100 for use with the system 10 of FIG. 1 formed according to an embodiment of the present invention. The bed or platform 100 is used to secure the patient 110 (FIG. 1) during treatment. The bed 100 is supported by a sturdy base frame 610 connected to a pedestal 630. The bed 100 may contain mechanisms that automate movement for ease of patient placement, such as an actuator that tilts the bed up and down. These mechanisms may be contained in the bed pedestal 630. The bed 100 may also contain an apparatus for recording patient weight such as a foot stand 620 for use when the bed is in the vertical, upwards tilted position. A comfortable mattress may be supported by a mattress base 640. The bed 100 may employ a split mattress design, containing both an upper mattress pad 650 and a lower mattress pad 660. In this example, the upper and lower mattresses 650 and 660 are pressed closely together near a point 670 where the lordotic region of the lumbar spine of the patient 110 (FIG. 1) lies on the bed 100. The automated functions of the bed 100 may assist patients who have difficulty positioning themselves on a normal bed.

A benefit of the decompression system 10 is that the system 10 can be used to determine the resistance of the spine to treatment, and can even be used to determine the resistance of non-para-spinal muscles separately from total spinal resistance. Referring to FIG. 1, the actuator 170 of the decompression system 10 is able to provide a measure of torque or power to move the tension strap 120 connected to the patient 110 a specific distance, and this work can be used to calculate the resistance of para spinal muscle contractions and the resistance of the spine itself to the elongation that occurs as the strap 120 is retracted or extended.

By gauging the work required to elongate the spine as described below, non-para-spinal muscle resistance can be approximated and accounted for. Once an approximation of the non-para-spinal muscle resistance is made, metrics such as tension-producing actuator work or applied tensile force (determined by the loadcell 150 in-line with the tension strap 120) can be used to calculate para-spinal muscle resistance. By this process, spinal elongation can be maximized as the intended spinal elongation profile for each patient 110 is adjusted immediately to resistances to the therapy. Thus, para-spinal muscle fatigue will increase as the thresholds at which these muscles constrict are closely monitored and not breached. Additionally, psychological para-spinal muscle constriction is decreased or eliminated because the therapy more closely tracks the natural responses of the spine in real-time.

For example, during treatment, spinal decompression forces applied to a region of the human spine will elongate that region. The spine, discounting para-spinal muscle activity, provides a resistance that can be modeled as a spring as:
Fₛ=Kₛ*Sₛ

Where Fₛ=force of spinal resistance which excludes para-spinal muscle activity; Kₛ=linear or non-linear spring constant (may be modeled as a lookup table); and Sₛ=distance (“spinal elongation”). Additionally, the spine can offer resistance via the para-spinal muscle contraction. Para-spinal muscle contraction is modeled as a resistant force: Fₚ. Thus the total resistance offered by the spine is:
Fₛ+Fₚ

In operation, the tension-producing actuator 170 or encoder 180 may communicate position, voltage or power applied to the actuator 170 to either or both of the actuator controller 192 and the computing device 190. The actuator controller 192 may monitor the aforementioned variables and may communicate these to the computing device 190. The encoder 180 communicates any number of tension-producing actuator variables, including but not limited to, position (i.e., distance the actuator 170 has traveled), power (voltage), speed or rate, acceleration, and direction to the actuator controller 192 and/or the computing device 190. The encoder 180 may be a distinct device or a part of the tension system 160 (or may use the loadcell 150 to estimate work). The encoder 180, feedback system 160, and actuator controller 192 all serve as part of a metric feedback system that tracks and communicates information such as power, distance, and tensile force between the actuator 170 and the computing device 190.

In one embodiment, the tension-producing actuator controller 192 may be the servo motor 230 (FIG. 2) that is connected to the spool 242 which carries the tension strap 120. This motor 230 is instructed to rotate the motor shaft 220 some distance which in turn tightens the tension strap 120 by the same distance:
Sₘ=sin(\acute{Ø})*2*pi*r
Where Sₘ=arc length, \acute{Ø}=angle difference in the actuator 170, and 2*pi*r=circumference of the actuator 170 and encoder 180 travel or the circumference of the tension strap spool 242. This movement is modeled as:
Sₘ=Sₛ
where the arc length of tension-producing actuator 170 travel is generally equivalent to the spinal elongation. Alternatively, spinal elongation (i.e., strap movement) may be measured in other ways, such as by optical monitoring devices that measure the movement of the strap. The stretch of the tension strap 120 is generally negligible and is removed from consideration. Alternatively, the formula can be adjusted to account for linear movement of the shaft 330 of the linear actuator of FIG. 3.

The work of the actuator 170 is modeled as:
Wₘ=Fₘ*t
where Wₘ=tension-producing actuator work, Fₘ=tension-producing actuator torque, and t=time of force application. Work equals force multiplied by time.

The force of the actuator 170 can be modeled as directly related to the voltaic potential or power applied to the actuator 170. Thus:
Vₘ=Fₘ
where Vₘ=voltage or power applied to the actuator 170. The force the actuator 170 applies is equivalent to that offered by the spine model:
Vₘ=Fₘ=Fₛ+Fₚ

It is important to note that if the encoder function does not provide the power applied by the actuator 170, then this information can be approximated by the tension measuring device 150 which can communicate the information either directly or indirectly to the computing device 190.

The following extends from the preceding models:
Wₘ=Fₘ*t
Wₘ=Vₘ*t
Wₘ=[Fₛ+Fₚ]*t
Wₘ=[(Kₛ*Sₛ)+Fₚ]*t
Wₘ=[(Kₛ*Sₘ)+Fₚ]*t
Wₘ=[(Kₛ*(sin(\acute{Ø})*2*pi*r))+Fₚ]*t
Thus, a regimen can be utilized such that the para-spinal muscle activity is directly related to the work performed by the actuator 170. An approximation of the resistance to spinal elongation that is not a result of para-spinal muscle activity works to more closely relate the work of the actuator to para-spinal muscle activity. Alternatively, the system 10 can be used simply to determine total resistance, without breaking resistance down into para-spinal and non-para-spinal muscle categories. The computer 190 receiving the metrics from the actuator 170, encoder 180 or actuator controller 192 can use the metrics to calculate para-spinal muscle resistance or total spinal resistance based on the calculations above and display them to the system user.

In another embodiment, a method utilized for approximating non-para-spinal muscle resistance to spinal elongation involves the bed 100 on which the patient 110 is lying. FIG. 7 illustrates an isometric view of the bed 100 of FIG. 6 according to an embodiment of the present invention. The lower mattress 660 of the bed 100 extends on shafts 720 from the upper mattress 650. The upper mattress 650 of the bed 100 remains fixed. In this example, the upper body of the patient 110 (FIG. 1) is secured to the upper mattress 650, which is in a fixed position. The lower mattress 660 is allowed to slide a certain distance with respect to the upper mattress 650 on the shafts 720 via bearings contained within pillow blocks of the upper and lower mattresses 650 and 660. This distance the lower mattress 660 travels is generally greater than that of the spinal elongation achieved during decompression therapy.

In operation, one of several methods is employed during the first spinal elongation cycles to estimate spinal elongation resistance that is not related to para-spinal muscle contraction. During this period, the lower mattress 660 remains fixed, pressed tightly against the upper mattress 650, and the distance the tension strap 120 (FIG. 1) is moved is minimal. The tension system 10 records the distance as related to the work involved in moving the strap 120, as discussed above. Resistance due to the spinal column can be approximated and extrapolated to increased spinal elongation distances. Once this approximation is made, the lower mattress 660 is allowed to ‘float’ along the shafts 720 away from the upper mattress 650 such that the patient's spine elongates further. Since the mattresses 660 and 650 are split at the point 670 (FIG. 6) underneath the lordotic region of the lumbar spine, the lower half of the body is positioned to allow further spinal elongation. At this point, additional resistance to spinal elongation not accounted for by the extrapolated resistance already calculated can be attributed to para-spinal muscle activity.

An alternative spinal elongation program may simply require a set limit to a variable such as measured tensile force or actual tension-producing actuator work and run a treatment for a specific or unspecified period of time. This simple treatment program would gradually and continually elongate the spine without cycling elongation, extending the strap or belt smoothly and slightly when measured tensile force or tension-producing actuator work is exceeded. The treatment program may then recalculate a smooth and steady path towards increasing spinal elongation. This simple treatment program would continuously engage the para-spinal muscle response and achieve maximum spinal elongation over a period of time.

Referring to FIG. 1, intervertebral discs are allowed to heal when intervertebral disc space is increased. As decompression therapy is a localized effect, there is a select region of the spine that can be elongated. Because the tension-producing actuator 170 provides the controller 192 with feedback that dictates distance (circumferential motor travel or linear distance in a linear actuator, for example) and that distance can be related to spinal column elongation, a method for spinal decompression that is distance-based and not actuator speed or rate-based is possible. The metric is a useful tool when made available for the healthcare provider. The spinal elongation metric can be used to track spinal column relaxation and accommodation to the therapy. Spinal elongation can be related additionally to applied tensile forces or tension-producing actuator work (e.g. voltage, torque, power, etc.). This relation can show how well a patient is responding to spinal elongation. This relation can be used to track a patient's progress over successive treatments. Limits placed on actuator work or on applied tensile force can lead to treatment and patient specific para-spinal muscle interaction in real-time. For example, as an intended spinal elongation profile is in progress (e.g. during a patient treatment) and an applied tensile force limit is neared, reached, or breached, the spinal elongation can be paused or reversed slightly, allowing para-spinal muscle relaxation. The spinal decompression device can then begin to return towards the intended spinal elongation distance while monitoring applied tensile forces which relate para-spinal muscle resistance.

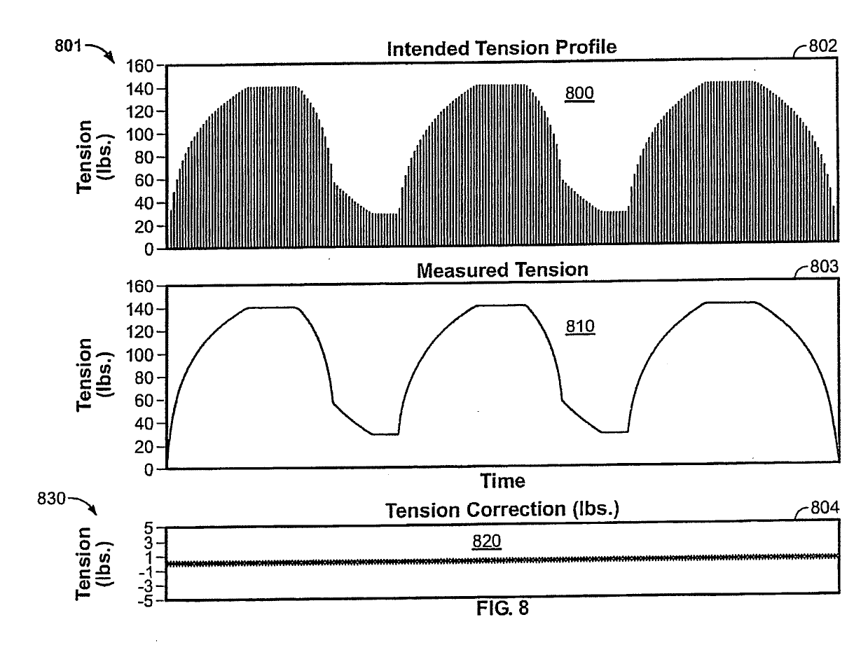

FIG. 8 illustrates a tension profile 801 for use in providing decompression therapy to a patient. The profile 801 includes first, second, and third charts 802, 803, and 804, respectively. The first chart 802 displays a pre-determined tension profile 800 for conventional spinal decompression systems showing tension in lbs. on the y-axis and time on the x-axis. Tension is applied to the spine of the patient following the parameters of the profile 800. The pre-determined tension profile 800 may be calculated based on patient body weight, patient therapy history, patient acceptance to the treatment, or other factors. Basic spinal decompression therapy systems utilizing a non-feedback-providing actuator typically implement a tension profile such as that shown in FIG. 8 via a tension measuring device such as a loadcell. Loadcell feedback is used to adjust the speed or rate of the non-feedback-providing tension-producing actuator which results in increased or decreased tensile force delivered to the patient. In the second chart 803, tension feedback 810 from the loadcell is plotted to demonstrate the system's adherence to the intended tension profile 800. In the second chart 803, the intended tension profile 800 is matched exactly by the loadcell tension feedback 810.

In the third 804 of the three charts, tension correction or adjustment 820 is plotted. A tension correction or adjustment graph 820 is typically plotted with a y-axis centered at zero and having plus and minus tensile force limits 830. In this example, the tension correction or adjustment graph 820 has tensile force correction limits 830 of plus or minus five pounds. Because the intended tension profile 800 of the first chart 802 is matched exactly by the loadcell feedback 810 of the second chart 803, the tension correction or adjustment graph 820 stays at zero pounds throughout the treatment.

Typically, a system has these force limits 830 hard-set such that tensile force correction will not accommodate corrections exceeding these limits 830. In the case where the measured loadcell feedback 810 is less or greater than the intended tension profile 800 by more than the hard-set tensile force correction limits 830, the intended tension profile 800 is not met. Therefore, the system controlling device that monitors and controls the treatment may stop the treatment.

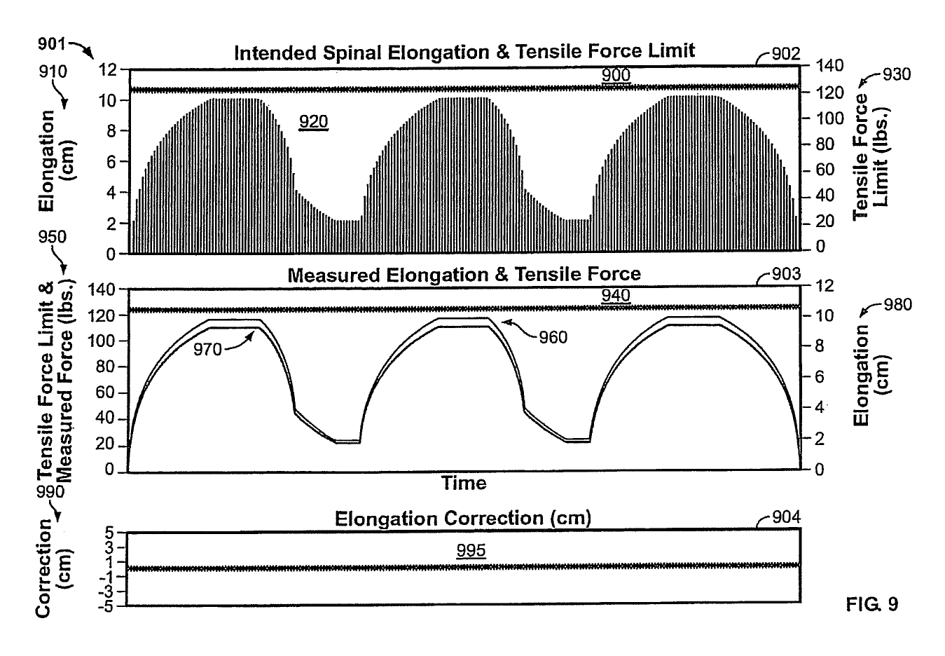

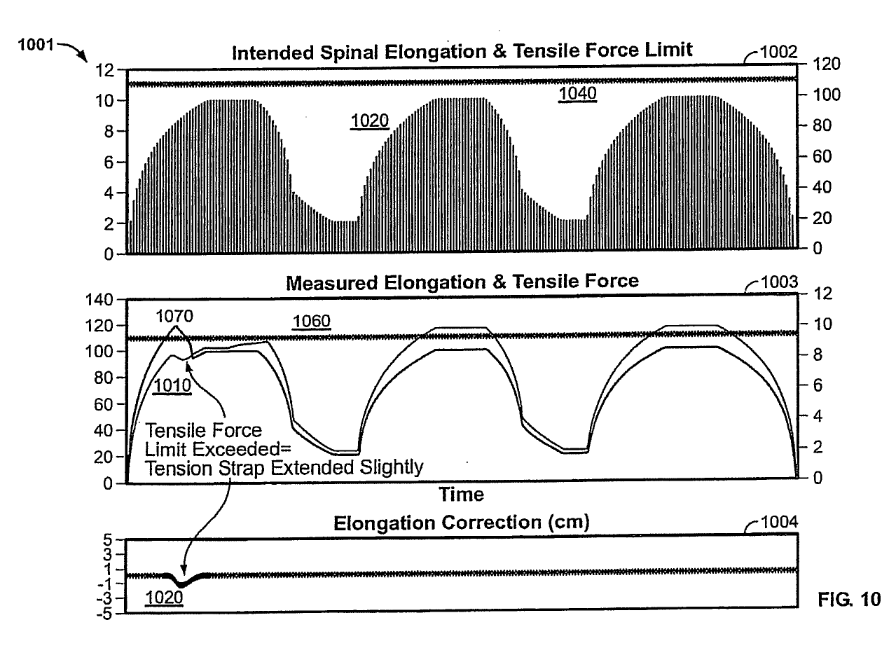

FIG. 9 illustrates a spinal elongation and tension profile 901 including first, second, and third charts 902, 903, and 904, respectively, formed according to an embodiment of the present invention. The profile 901 is used with the decompression therapy system 10 (FIG. 1) utilizing a distance-based methodology for spinal decompression. Alternatively, the profile 901 may be configured for use with a traction therapy system. In the first chart 902, the intended spinal elongation profile 920 for spinal decompression is displayed on a monitor of the computer 190 (FIG. 1) and shows the distance that the patient interface device 120 (FIG. 1), is intended to be extended or retracted over the course of a treatment. In this case the intended spinal elongation profile 920 is shown in centimeters (cm) and is plotted on the left y-axis 910 with time on the x-axis. By way of example only, the intended spinal elongation profile 920 extends from zero to about ten centimeters, and is plotted as a continuous smooth function with no abrupt changes in elongation. On the same first chart 902, above the spinal elongation profile 920, the tensile force limit 900 is plotted in lbs. on the right y-axis 930. In this treatment profile, the tensile force limit 900 is plotted as a continuous 125 pound limit. However, this limit 900 may be set to change over the course of the treatment, and may match somewhat the shape of the intended spinal elongation profile 920. The profile of the first chart 902 is implemented and monitored to treat the patient through the therapy system 10 by way of the computer 190 (FIG. 1).

Over the course of a treatment using the decompression therapy system 10 of FIG. 1, the intended elongation 920 may be plotted against the actual elongation 960. In the second graph 903, the actual elongation 960, which may be measured by the encoder device 180 (FIG. 1) as discussed above, is communicated to the computer 190 and plotted, and matches closely the intended spinal elongation profile 920. The right y-axis 980 of the second chart 903 shows actual spinal elongation in centimeters. On the same graph, the actuator work or tensile force may be monitored and plotted 970 and used as a gauge to adjust the amount of strap extension or contraction. In this example, tensile force is measured by the loadcell 420 (FIG. 4), and is monitored to ensure that applied tensile force does not exceed the tensile force limit 940. In this example, the tensile force limits of the first and second charts, 900 and 940, respectively, are the same plot. In the second graph 903, the tensile force limit 940 is plotted on the left y-axis 950. The measured tensile force 970 is also plotted on the left y-axis 950. In this example, the measured spinal elongation 960 of the second chart 903 matches closely the intended spinal elongation profile 920 of the first chart 902, and the measured tensile forces 970 do not exceed the tensile force limits 940 at any point during the treatment.

In the third chart 904 of profile 901, a spinal elongation correction 995 graph is plotted on the left y-axis 990 at zero centimeters (cm) throughout the entire treatment because the intended spinal elongation profile 920 of the first chart 902 and the measured spinal elongation 960 of the second chart 903 match closely and the measured tensile forces 970 do not exceed the tensile force limits 940. The third chart 904 includes spinal elongation correction limits 990 set at plus and minus five centimeters (cm) at the left y-axis.

In one embodiment the treatment program may be programmed to correct spinal elongation by extending the tension strap 120 (FIG. 1) so as to reduce resultant tension delivered to the patient 110 (FIG. 1) where the actual elongation 960 exceeds the intended elongation 920. In that embodiment, the treatment program is intended to ensure that the spine 108 (FIG. 1) is elongated according to the intended spinal elongation profile 920. In another embodiment, the treatment program may be intended to exceed the spinal elongation profile 920 under such conditions as measured tensile forces 970 are lower by a certain percentage than the tensile force limits 900 and 940. In this scenario, the patient may be particularly relaxed and responsive to spinal decompression during that specific treatment period. The treatment program may then retract the strap 120 which further elongates the spine 108 and increases the decompression therapy benefit to the patient. In that case, the measured spinal elongation 960 would exceed, for a certain period of the treatment profile, the intended spinal elongation profile 920. Alternatively, the strap 120 could be released if the actual tensile force 970 exceeded the force limits 900 and 940. Thus, the therapist can track the patient's para-spinal muscle interactions in real-time, and the system 10 operates as a patient-specific decompression system. After treatment, this data may then be saved or plotted and reviewed during the patient's next treatment period. A healthcare provider may choose to increase the intended spinal elongation profile 920 upper limits based on the past treatment records.

In the case where the treatment program is intended to exceed the intended spinal elongation profile 920, the method of the present invention provides for patient specific spinal decompression in the event that the patient is particularly responsive to spinal elongation during a specific treatment period.

Additionally, the patient may be able to view the spinal elongation and tension profile 901 on a screen or monitor during treatment and watch the actual elongation be charted on the second chart 903 against the tension limit 940 and intended elongation profile 920. Where the patient observes that the intended elongation profile 920 is not being met, the patient can consciously relax the spinal muscles to facilitate further elongation and decompression. Thus, the patient can interact with the profile to provide biofeedback such that the treatment is more patient specific.

FIG. 10 illustrates a spinal elongation and tension profile 1001 including first, second, and third charts 1002, 1003, and 1004, respectively, formed according to an embodiment of the present invention. The profile 1001 is used with the decompression therapy system 10 (FIG. 1) utilizing a distance-based, spinal elongation methodology. Alternatively, the profile 1001 may be configured for use with a traction therapy system. In this example the intended spinal elongation profile 1020 and a tensile force limit 1040 are plotted in the first chart 1002. The tensile force limit 1040 is set to 110 pounds.

In the second chart 1003, the measured tension 1070 increases and begins to exceed the preset tensile force limit 1060. This data is interpreted to mean that the patient's para-spinal muscles are resisting spinal elongation for any number of reasons including, but not limited to, the psychological (claustrophobia, panic, etc.), the reflexive (too rapid a change, or uncomfortable or incorrect patient positioning), or the physiological (tight muscles possibly from excessive exertion prior to treatment). Possibly, the exact same intended spinal elongation profile implemented during a previous session might not have had an instance of exceeding the tensile force limit 1060. Because the condition of the patient 110 (FIG. 1) is in a constant state of flux, a distance-based spinal elongation methodology with various forms of tension-producing actuator feedback and control makes patient-specific therapy possible.

In this example, as the measured tensile forces 1070 exceed the predetermined tensile force limits 1060, the actuator 170 (FIG. 1) is instructed by the computer 190 (FIG. 1) to extend the tension strap 120 (FIG. 1) by a specific or non-specific distance. As the strap 120 is extended, the measured tensile force 1070 is compared to the predetermined tensile force limit 1060. This cycle continues until the measured tensile forces 1070 are below the tensile force limit 1060.

In the third chart 1004, at the point where the measured tensile forces 1070 of the second chart 1003 exceed the predetermined tensile force limit 1060, the chart 1004 shows the spinal elongation correction 1020, or an extension of the tension strap 120 (FIG. 1), is about one and one-half centimeters. At the same time, the measured spinal elongation 1010 in the second graph 1003, as may be provided by the encoder device 180 (FIG. 1), is shown to be reduced slightly.

Once the measured tensile forces 1070 are brought under the predetermined tensile force limits 1060, the treatment program may calculate a safe, slower rate of spinal elongation 1020 that brings the patient's spinal elongation in line with the intended spinal elongation shown in the first graph 1002. By this method, the patient's spine 108 (FIG. 1) is constantly engaged to keep para-spinal muscles at or near their reflexive contraction points. The patient 110 (FIG. 1) psychologically is continuously less aware of the spinal elongation as there are less signals registering para-spinal reflexive muscle contraction. Throughout the treatment, the spine 108 is elongated to the maximum amount such that the para-spinal muscle contraction and corresponding psychological para-spinal muscle contraction is reduced. This method provides improved spinal decompression benefit and improved patient safety.

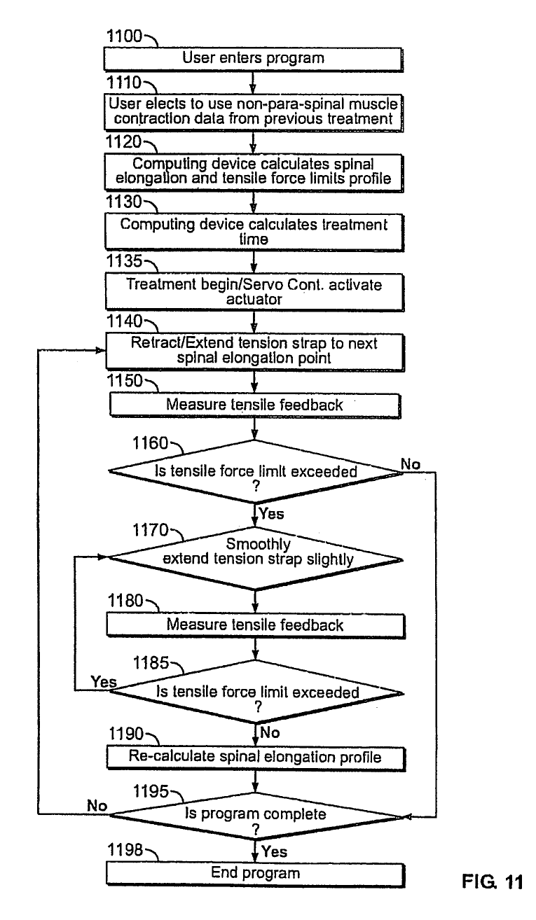

FIG. 11 illustrates a flowchart demonstrating an algorithm for implementing patient specific spinal decompression therapy using a distance-based spinal elongation methodology formed according to an embodiment of the present invention. In this program, the user enters the treatment profile data for the current patient 1100. This data may include patient weight, treatment time, upper tensile force or tension-producing actuator work, spinal elongation correction limits, or other factors.

The user may decide on treatment values based on previous treatment history. In this case, the user elects to use data from a previous treatment that demonstrated non-para-spinal muscle resistance to spinal elongation 1110. This data may reduce or eliminate the need for the user to spend several minutes approximating non-para-spinal muscle resistance to spinal elongation.

The computing or controlling device then calculates a predetermined spinal elongation profile 1120 and may communicate this information to a tension-producing actuator controller and/or tension-producing actuator. The computing device is able to calculate the total treatment time 1130 which may be made available to the healthcare provider and patient.

Once the predetermined spinal elongation program is calculated and all variables are set, the treatment is begun 1135. The tension-producing actuator is instructed to extend or retract the patient interface device by a specific distance to the first ‘point’ in the predetermined spinal elongation profile 1140.

Once the strap is moved, the program measures the limiting variable or variables, in this case the tensile force measured by the loadcell configuration 1150. This metric is compared to the preset tensile force limits for the treatment period 1160. If the measured tensile force limit is not exceeded, the program decides if the treatment is completed or if therapy is to continue 1195. If the treatment is not completed, the program proceeds to extend or retract the strap a specific distance to the next ‘point’ in the predetermined spinal elongation profile 1140. If the measured tensile force limit of 1150 does exceed the preset tensile force limits for the treatment period 1160, then the program proceeds to extend the strap a specific amount in a smooth fashion 1170. After this incremental extension, the tensile force is again measured 1180. If the measured tensile force is again determined to be above the preset tension limits 1185, then the smooth incremental strap extension 1170 is repeated.

If however the measured tensile force is determined to be less than the preset tensile force limits 1185, the program proceeds to calculate a smooth and steady increase in spinal elongation that will return the patient to the intended spinal elongation profile 1190.

The program is then directed to determine if the treatment period is complete 1195. If the treatment period is not completed, the program extends or retracts the belt a specific distance 1140 to the next ‘point’ of the predetermined spinal elongation profile. If the program determines the treatment period is complete, the program is ended 1198.

Spinal elongation is a more appropriate metric for determining the progress of decompression therapy than conventional systems monitoring only tension-providing actuator speed or rate. The spinal elongation profiles of the different embodiments of the present invention can be recorded and recalled and can show exactly what kind of spinal elongation the patient accomplished during a previous therapy session. Using records from conventional tension monitoring systems, the therapist could only determine what amounts of tension were applied to the patient. With the elongation recordings of the present invention, the therapist can determine the progress of the patient during the course of decompression therapy. For example, the recording from the first treatment may show that at several times the patient's spine was unable to reach intended elongation due to para-spinal muscle resistance while recordings from subsequent treatment show that the para-spinal muscles of the patient's spine resisted less and eventually the patient's spine reached or exceeded the intended elongation.

Furthermore, the patient's progress may be recorded as a percentage of the intended elongation. For example, the percentage of actual elongation measured against the intended elongation may increase as a percentage over successive treatments. A first treatment may result in an actual elongation graph covering only 85% of the area under the intended elongation graph. Successive treatments may result in a steady increase of the percentage of actual elongation relative to intended elongation. Subsequent treatments may increase to 90% to 95% to 100% to percentages where the actual elongation exceeds the intended elongation, such as 105%. By tracking the patient's progress as a percentage of the intended treatment, the patient-specific decompression therapy system of the present invention provides the therapist a gauge for determining the success of the decompression therapy.

In an alternative embodiment, spinal elongation and other metrics are not limited to use with decompression therapy but may be used to measure the progress of traditional traction therapy or any number of other methods where the application of tensile forces to the spine can be measured and recorded as a function of another metric such as elongation or work.

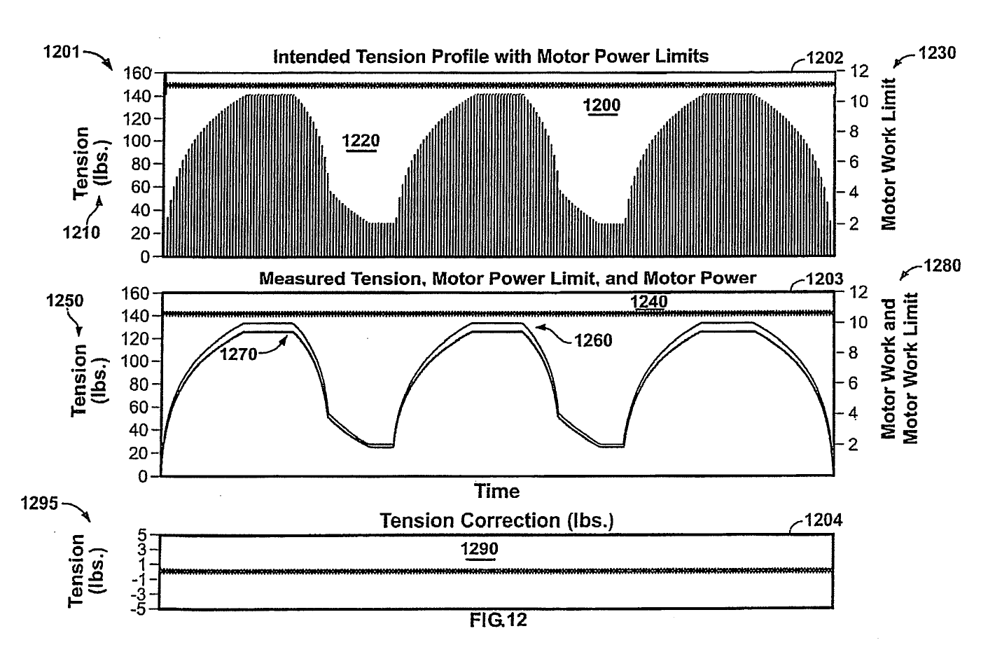

FIG. 12 illustrates a tension profile 1201 for spinal decompression that also tracks and provides biofeedback to the patient in the form of actuator or motor work. The spinal tension profile 1201 includes first, second, and third charts 1202, 1203, and 1204, respectively, formed according to an embodiment of the present invention. The profile 1201 is used with the decompression therapy system 10 (FIG. 1) utilizing a tension-based methodology for spinal decompression. Alternatively, the profile 1201 may be configured for use with a traction therapy system. In the first chart 1202, the intended spinal tension profile 1220 for spinal decompression is displayed on a monitor of the computer 190 (FIG. 1) and shows the tension that the patient interface device 120 (FIG. 1) is intended to apply to the patient over the course of a treatment. By way of example, the intended spinal tension profile 1220 is shown in pounds and is plotted on the left y-axis 1210 with time on the x-axis. The intended spinal tension profile 1220 extends from zero to about 160 pounds (lbs.), and is plotted as a continuous smooth function with no abrupt changes in tension. On the same top graph 1202, above the spinal tension profile 1220, the actuator work or power limit 1200 is plotted in using a relative scale on the right y-axis 1230. By way of example, the tension producing actuator 170 (FIG. 1) is a motor that performs work. In the treatment profile 1201, the motor work or power limit 1200 is plotted as a continuous limit. However, this limit 1200 may be set to change over the course of the treatment, and may match somewhat the shape of the intended spinal tension profile 1220. The profile of the first chart 1202 is implemented and monitored on the patient through the therapy system 10 by way of the computer 190 (FIG. 1).

Over the course of a treatment using the decompression therapy system 10 of FIG. 1, the intended tension 1220 may be plotted against the actual tension 1260 as shown in the second chart 1203. In the second graph 1203, the motor power or work limit 1240 is measured on the right y-axis 1280 and the measured tensile force is measured on the left y-axis 1250. The actual applied tensile force 1260 is plotted on the second chart 1203. The actual tension 1260, which may be measured by the loadcell 160 (FIG. 1) as discussed above, is communicated to the computer 190 and plotted, and matches closely the intended spinal tension profile 1220 of the first chart 1202. The motor power or work limit 1240 is plotted on the second chart 1203 and is the same as the motor power or work limit 1200 of the first chart 1202. Actual applied motor power or work 1270 is measured by either or both the encoder 180 and servo-amplifier 192 (FIG. 1) and calculated as discussed above, and is plotted on the second chart 1203. By way of example only, in the second chart 1203 the applied motor work 1270 does not exceed the motor work limit 1240 at any point during the treatment. The actual motor power or work 1270 is monitored to ensure that actual applied motor power or work 1270 does not exceed the motor power or work limit 1240. Thus, the second chart 1203 may be used as an additional gauge to adjust the amount of strap extension or contraction based on whether the actual motor power or work 1270 exceeds the work limit 1240.

In the third chart 1204 of profile 1201, a spinal tension correction 1290 graph is plotted on the left y-axis 1295 at zero pounds throughout the entire treatment because the intended spinal tension profile 1220 of the first chart 1202 and the measured spinal tension 1260 of the second chart 1203 match each other closely and the measured motor power or work 1270 does not exceed the motor power or work limits 1240. The third chart 1204 includes spinal tension correction limits 1290 set at plus and minus five pounds (lbs.) at the left y-axis.

In one embodiment the treatment program may be programmed to correct spinal tension by extending the tension strap 120 (FIG. 1) so as to reduce resultant tension 1260 delivered to the patient 110 (FIG. 1) where the actual spinal tension 1260 exceeds the intended spinal tension 1220. In that embodiment, the treatment program is intended to ensure that the tension is applied according to the intended spinal tension profile 1220. In another embodiment, the treatment program may be intended to exceed the spinal tension profile 1220 under such conditions where measured motor power or work 1270 is lower by a certain percentage than the motor work or power limits 1200 and 1240. In this scenario, the patient may be particularly relaxed and responsive to spinal decompression during that specific treatment period. The treatment program may then retract the strap 120 which further elongates the spine 108 and increases the decompression therapy benefit to the patient. In that case, the measured spinal tension 1260 would exceed, for a certain period of the treatment profile, the intended spinal tension profile 1220. Alternatively, the strap 120 could be released if the motor power or work limits 1200 and 1240 were exceeded. Thus, the therapist can track the patient's para-spinal muscle interactions in real-time, and therefore the system 10 operates as a patient-specific decompression system. After treatment, this data could then be saved or plotted and reviewed during the patient's next treatment period. A healthcare provider may choose to increase the intended spinal tension profile 1220 upper limits based on the past treatment records.

In the case where the treatment program is intended to exceed the intended spinal tension profile 1220, the method of the present invention provides for patient-specific spinal decompression in the event that the patient is particularly responsive to spinal tension during a specific treatment period.

Additionally, the patient may be able to view the charts 1202, 1203, and 1204 of the profile 1201 on a screen or monitor during treatment, such as on the monitor of the computer 190 (FIG. 1), and watch the actual tension 1260 be charted against the motor work or power limit 1240 and intended tension profile 1220. Where the patient observes that the intended tension profile 1220 is not being met, the patient can consciously relax the spinal muscles to facilitate further tension and decompression. Thus, the patient can interact with the profile to provide biofeedback such that the treatment is more patient-specific.

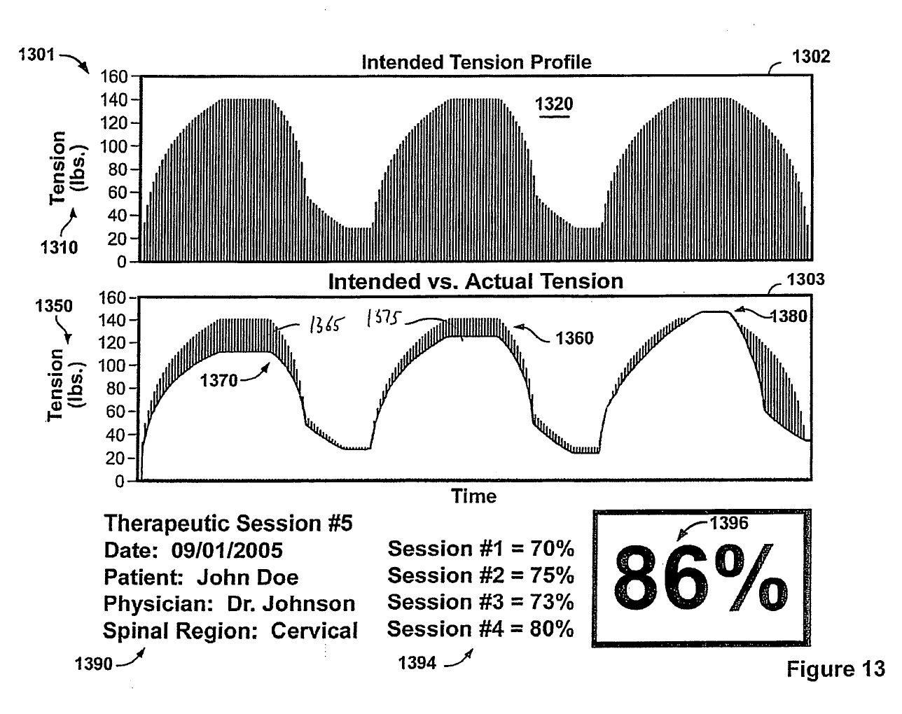

FIG. 13 illustrates a tension profile 1301 for spinal therapy formed according to an embodiment of the present invention. The spinal tension profile 1301 includes first and second charts 1302 and 1303. The profile 1301 is used with the traction or decompression therapy system 10 (FIG. 1) utilizing a tension-based methodology for spinal decompression and provides analysis of the treatment session. In the first chart 1302, the intended spinal tension profile 1320 for spinal decompression is displayed on a monitor of the computer 190 (FIG. 1) and shows the tension 1320 that the patient interface device 120 (FIG. 1) is intended to apply to the patient over the course of a treatment. By way of example, the intended spinal tension profile 1320 is shown in pounds and is plotted on the left y-axis 1310 with time on the x-axis. The intended spinal tension profile 1320 extends from zero to about 160 pounds (lbs.), and is plotted as a continuous smooth function with no abrupt changes in tension. The profile of the first chart 1302 is implemented and monitored on the patient through the therapy system 10 by way of the computer 190.

In the second chart 1303, the intended tension 1360 is plotted against the actual tension 1370 applied during the treatment. The intended tension 1360 of the second chart 1303 is generally the same as the intended tension 1320 of the first chart 1320. By way of example only, the title 1390 of the treatment session for which the profile 1301 is used is “Therapeutic Session #5”. In the second chart 1303, the actual tension 1370, which may be measured by the loadcell device 160 (FIG. 1), is communicated to the computer 190 (FIG. 1), and plotted, and matches closely the intended spinal tension profile 1360. The left y-axis 1350 of the second chart 1303 measures the actual tension 1370. The plots of the second chart 1303 are based on, as described above, algorithms used to measure both tension producing actuator work or power, as in the case of a servo motor and servo amplifier, and measured actual tension. In first and second tension peaks 1365 and 1375, respectively, of the second chart 1303, the actual tension 1370 is less than the intended tension 1360, so the intended tension 1360 was not achieved at these points in the treatment. Thus, motor work or power was close to, or exceeded, motor work or power limits during that treatment, and the motor was directed not to produce tension that required motor work or power over the motor work or power limits. In a third tension peak 1380, actual tension 1370 exceeded intended tension 1360, indicating motor work/power was sufficiently below preset motor work/power limits, allowing the computer 190, by use of the algorithms, to advance tension levels and increase decompression forces over that indicated by the intended tension profile 1360.

Data from the treatment is compiled and shown at the bottom of the profile chart 1301 at displays 1390, 1394, and 1396 for the patient and physician to view on the screen of the computer 190 (FIG. 1). Patient data display 1390 shows, by way of example only, the title of the treatment session, the date of the treatment session, the patient's name, the physician's name, and the spinal region treated during the treatment, such as the cervical spinal region. Patient data display 1396 shows a percentage, by way of example only, 86%, derived from the data plotted in the second chart 1303. This data is taken as the normalized area under the plot of the intended tension 1360 divided by the area under the plot of the actual or measured tension profile 1370. The division yields, in this example, 0.86, or 86%. Patient data display 1394 shows similar percentages based on calculations from past treatment sessions numbers 1 through 4. Thus, the patient is shown to progressively achieve actual tension plots 1370 more closely matching the intended tension plots 1360 with each successive treatment. In treatment #5 as shown in profile 1301, the patient achieved a tension compliance rate of 86%.

The physician may interpret the percentages shown in data displays 1394 and 1396 to mean that the patient has psychologically and physiologically adapted to the therapy, thus increasing the benefit of the therapy. It is possible to achieve a percentage of greater than 100%, in cases where the patient is especially receptive to the therapy.

The method whereby the algorithms are used to monitor and control tension can also be used to monitor additional metrics as described in this invention, such as motor power as discussed above. Thus, an operator can use the system 10 (FIG. 1) to determine tension levels that best suit the patient during a particular treatment, on a particular day, to increase the safety of the treatment and to benefit the patient. On a given day of treatment, the patient may be following the instructions of the physician and have increased flexibility and healing and be more able to achieve decompression benefit from the intended tension profile. Conversely, on another day, the patient may have lifted a particularly heavy object and not be as receptive to the intended tension profile. In any event, the methods of multiple variable monitoring during treatment, which may include but are not limited to motor power or work, tension, and spinal elongation, allow real-time insight to a particular treatment that is not otherwise available to the physician at the time of initial programming. This method of post-analysis of data allows the physician to more accurately program the traction or decompression device for the individual patient. In this example, a progressively increasing percentage shown in data displays 1394 and 1396 may indicate the physician should increase the intended tension profile 1320 during the next treatment. The simple percentage also can be used to relate relative success to the patient, encouraging the patient towards the goals of rehabilitation.

Furthermore, the advantages of relating tension-producing actuator work to para-spinal muscle response are many. Para-spinal muscle response is reflexively a safeguard against the damaging of the spine due to unnatural movement (i.e., excessive or overly-rapid stretch). The patient specific decompression therapy system of the present invention allows a therapist to track para-spinal muscle response both during a treatment and across a patient's treatment history. For example, the system allows the therapist to “spot” a para-spinal reflexive contraction during treatment when the actual elongation or tension does not match the intended elongation or tension, respectively. The therapist can then reverse the spinal elongation or tension slightly, such that the contraction is decreased and then the therapist can continue the decompression therapy. By continuously tracking para-spinal muscle activity as provided for in the embodiments of the present invention, reflexive and psychological para-spinal muscle contraction can be reduced. Also, by tracking para-spinal muscle activity and spinal elongation, measures of work or applied tensile forces can be attributed to treatment success.

Additionally, advancement past the traditional limitations of either traction-based or spinal decompression systems can be achieved by monitoring actual work (or other related parameter) required to perform a tension profile on a patient. In this way, patient compliance with a treatment can be compared across therapy sessions (more work to achieve the same tension profile may indicate limited patient compliance to a particular treatment). If this metric is provided in real-time to the patient during a treatment, then the patient can observe and voluntarily relax during times of stress. Moreover, spinal elongation may be provided and recorded for additional analysis. A metric comparing intended spinal elongation and/or tension profile to actual elongation and/or tension profile, respectively, can be created to show actual patient compliance with a treatment as a percentage of the intended patient treatment over the course of many treatments.

In all systems described above, regardless of traction-based or decompression-based modalities, recorded parameters are displayed for the patient in such a way as to provide biofeedback and increase patient benefit. Through the recorded parameters, technicians can more accurately track patient progress and adapt spinal treatment to the patient's specific requirements. In real-time during the treatment the system can adapt to the patient and the patient can be adapted to the treatment, whereupon patient-specific spinal therapy is achieved. Through the use of these metrics (such as, by way of example only, elongation, mark, distance, voltage, power) and their derivatives, a specific figure or set of figures can be created as a standard for determining whether a patient's compliance with intended treatment is a therapeutic success.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A system for applying a treatment profile during spinal therapy, said system, comprising:
a control system for implementing a spinal treatment profile;
an actuator for producing a tensile force based on the implementation of said spinal treatment profile by said control system;
a patient interface device configured to apply the tensile force from said actuator to the spine of a patient; and
a feedback system configured to provide metrics resulting from the application of the tensile force to the spine to said control system such that said control system calculates elongation of the spine of the patient as a result of the tensile force applied to the spine and monitors the elongation relative to an intended elongation program of said treatment profile.

2. The system of claim 1, wherein said control system calculates the tensile force applied by said actuator during application of said treatment profile and monitors said applied tensile force relative to a predetermined tensile force limit of said treatment profile.

3. The system of claim 2, wherein said control system displays said treatment profile on a monitor showing charts plotting the elongation, the intended elongation program, the applied tensile force, and the tensile force limit, whereby the patient may watch the charts during therapy and provide biofeedback to the treatment.

4. The system of claim 3, wherein said control system further displays the elongation as a percentage of the intended elongation program.

5. The system of claim 1, wherein the tensile force is applied to the spine of the patient in a plurality of cycles defined in said treatment profile.

6. The system of claim 2, wherein said control system displays said treatment profile on a monitor showing a first chart plotting the tensile force applied by said actuator and the tensile force limit, whereby when the plotted applied tensile force exceeds the plotted tensile force limit, said control system adjusts the tensile force applied by said actuator to the spine of the patient until the plotted applied tensile force no longer exceeds the tensile force limit.

7. The system of claim 1, wherein said control system calculates spinal resistance to the tensile force applied during therapy based on at least one of such variables provided by said feedback system as tensile force applied by said actuator, distance traveled by said actuator, and work performed by said actuator during therapy.

8. The system of claim 1, further including a bed having a first section and a second section, whereby said second section is configured to move away from said first section during therapy in order to further elongate the spine of the patient.

9. A system for applying a treatment profile during spinal therapy, said system, comprising:
a control system for implementing a spinal treatment profile;
an actuator for producing a tensile force based on the implementation of said spinal treatment profile by said control system;
a patient interface device configured to apply the tensile force from said actuator to the spine of a patient; and
a feedback system configured to provide metrics resulting from the application of the tensile force to the spine to said control system such that said control system calculates work performed by said actuator during application of said treatment profile and monitors the performed work relative to predetermined work limit of said treatment profile.

10. The system of claim 9, wherein said control system monitors the actual tensile force applied to the spine of the patient relative to an intended tensile force program of said treatment profile.

11. The system of claim 10, wherein said control system displays said treatment profile on a monitor showing charts plotting the performed work, the predetermined work limit, the actual tensile force applied to the spine, and the intended tensile force program, whereby the patient may watch the charts during therapy and provide biofeedback to the treatment.

12. The system of claim 11, wherein said control system further displays the actual tensile force as a percentage of the intended tensile force program.

13. The system of claim 9, wherein the tensile force is applied to the spine of the patient in a plurality of cycles defined in said treatment profile.

14. The system of claim 9, wherein said control system displays said treatment profile on a monitor showing a first chart plotting the performed work and the predetermined work limit, whereby when the plotted performed work exceeds the plotted predetermined work limit, said control system adjusts the tensile force applied by said actuator to the spine of the patient until the plotted performed work no longer exceeds the predetermined work limit.

15. The system of claim 9, wherein said control system calculates spinal resistance to the tensile force applied during therapy based on at least one of such variables provided by said feedback system as tensile force applied by said actuator, distance traveled by said actuator, and work performed by said actuator during therapy.

16. The system of claim 9, further including a bed having a first section and a second section, whereby said second section is configured to move away from said first section during therapy in order to further elongate the spine of the patient.

17. A system for applying a treatment profile during spinal therapy, said system, comprising:
a control system for implementing a spinal treatment profile;
an actuator for producing a tensile force as defined in said treatment profile based on commands from said control system;
a patient interface device configured to apply the tensile force from said actuator to the spine of a patient; and
a feedback system configured to provide metrics resulting from the application of the tensile force to the spine to said control system, wherein said treatment profile includes a limit based on a metric related to the application of the tensile force to the spine such that, when the application of the tensile force to the spine results in the metric exceeding the limit, said control system reduces the tensile force applied by said actuator to the spine.

18. The system of claim 17, wherein the limit is for one of the tensile force applied by said actuator and work by said actuator in applying the tensile force.

19. A method of applying a treatment profile for spinal therapy, said method including:
providing a tension-producing actuator;
providing a control system that implements the treatment profile to control the actuator;
applying a tensile force to the spine of a patient in accordance with the treatment profile;
calculating the elongation of the spine of the patient resulting from the application of the tensile force; and
monitoring and adjusting the application of the tensile force applied to the spine of the patient with the control system based on the calculations of spinal elongation during therapy.

20. A method of applying a treatment profile for spinal therapy, said method including:
providing a tension-producing actuator;
providing a control system that implements the treatment profile to control the actuator;
applying a tensile force to the spine of a patient with the actuator in accordance with the treatment profile;
calculating the work performed by the actuator in applying the tensile force to the spine of the patient; and
monitoring and adjusting the application of the tensile force applied to the spine of the patient with the control system based on the calculations of work performed by the actuator during the spinal therapy.
